(19) United States
(12) Patent Application Publication
(10) Pub. No.: US 2025/0204814 A1
(43) Pub. Date: Jun. 26, 2025

(54) MIXED CONDUCTING, INTRINSICALLY STRETCHABLE ENZYME MEMBRANES AND METHODS OF FABRICATION

(71) Applicant: The University of Hong Kong, Hong Kong (HK)

(72) Inventors: Shiming ZHANG, Hong Kong (HK); Binbin CUI, Hong Kong (HK); Dingyao LIU, Hong Kong (HK); Jing BAI, Hong Kong (HK); Xinyu TIAN, Hong Kong (HK)

(21) Appl. No.: 18/818,607

(22) Filed: Aug. 29, 2024

Related U.S. Application Data
(60) Provisional application No. 63/612,381, filed on Dec. 20, 2023.

(51) Int. Cl. A61B 5/145; A61B 5/00; A61B 5/1477; C12N 9/04; C12N 11/04

(52) U.S. Cl. CPC A61B 5/14532; A61B 5/0002; A61B 5/1477; A61B 5/742; A61B 5/746; C12N 9/0006; C12N 11/04

(57) ABSTRACT

A stretchable, conducting, and redox-active hydrogel with an interpenetrating double-network structure is provided. This structure is formed by infiltrating a brittle pure-gel conducting hydrogel with a stretchable hydrogel. Ferrocene derivatives are immobilized on the chains of the stretchable hydrogel through covalent bonds, and glucose oxidases are crosslinked to the stretchable hydrogel using a room-temperature crosslinker.

### CROSS-REFERENCE TO RELATED APPLICATIONS

The present application claims priority from U.S. provisional patent application Ser. No. 63/612,381 filed Dec. 20, 2023, and the disclosure of which is incorporated herein by reference in its entirety.

### FIELD OF THE INVENTION

The present invention generally relates to the medical equipment field. More specifically the present invention relates to stretchable, conducting, and redox-active hydrogels for glucose monitoring.

### BACKGROUND OF THE INVENTION

The evolution of biosensors, initiated by Leland Clark's invention of the first glucose biosensor in 1962, has witnessed significant progress. Biosensors are essential bio-analytical tools that convert biological reactions into quantifiable signals, consisting of three core components: a biological sensing element, a detector or transducer unit, and a signal processing system. In the context of glucose biosensors, the sensing element is the enzyme membrane.

A promising frontier for biosensors lies in the medical field, where these devices play a pivotal role in diagnosing, monitoring, and treating various health conditions. Wearable biosensors, enabling continuous health monitoring, provide an alternative to traditional hospitalization and constant patient supervision. For example, continuous glucose monitoring systems (CGMs) have greatly improved the quality of life for individuals with diabetes, offering mobility and a constant stream of diagnostic data. However, existing needle-dependent CGMs cause discomfort and can lead to bleeding issues.

To address this, ongoing research is shifting towards non-invasive CGMs, utilizing skin-conformable, intrinsically stretchable medical tapes or bandages to develop stretchable glucose sensors. Yet, the enzyme membranes, responsible for catalyzing glucose into detectable currents in these sensors, are primarily composed of rigid and fragile polymers. Despite their importance, stretchable enzyme membranes remain scarce.

Recent advances in stretchable and multifunctional hydrogels offer a potential solution for developing stretchable enzyme membranes. These hydrogels are unique materials that can combine inherently and mutually exclusive properties, including electrical, mechanical, redox, and diffusive properties, all of which are essential for achieving enzyme functionality.

Therefore, the present invention addresses the fragility of existing enzyme membranes and aims to provide skin-conformable, soft, and stretchable glucose sensors that can be integrated into commercial stretchable sweat patches or wound- healing bandages.

### SUMMARY OF THE INVENTION

It is an objective of the present invention to provide a device, material, or method to solve the aforementioned technical problems.

In accordance with the first aspect of the present invention, a stretchable, conducting, and redox-active hydrogel is provided. Particularly the hydrogel includes:
an interpenetrating double-network structure, including:
   a brittle pure-gel conducting hydrogel; and
   a stretchable hydrogel, wherein the stretchable hydrogel infiltrates the brittle pure-gel conducting hydrogel to form the interpenetrating double-network structure;
   wherein ferrocene derivatives are immobilized on chains of the stretchable hydrogel via covalent bonds and glucose oxidases are crosslinked to the stretchable hydrogel utilizing a room-temperature crosslinker.

In accordance with one embodiment of the present invention, the hydrogel remains conductive and maintains redox properties after stretching up to 200% of the initial length.

In accordance with one embodiment of the present invention, the brittle pure-gel conducting hydrogel network is poly(3,4-ethylenedioxythiophene) polystyrene sulfonate (PEDOT: PSS).

In accordance with one embodiment of the present invention, the stretchable hydrogel is selected from polyacrylamide (PAAm), poly(acrylic acid) (PAAc), or gelatin methacryloyl (GelMA).

In accordance with one embodiment of the present invention, the room-temperature crosslinker is polyethylene glycol diglycidyl ether (PEGDE).

In accordance with one embodiment of the present invention, the ferrocene derivatives include ferrocenium ion, ferrocene carboxylic acid, ferrocene methanol, ferrocenylmethyl trimethylammonium, ferrocene boronic acid, and ferrocene dimethylamine.

In accordance with one embodiment of the present invention, the brittle pure-gel conducting hydrogel has a porosity ranging from 20% to 90%.

In accordance with a second aspect of the present invention, a method of fabricating a stretchable, conducting, and redox-active hydrogel is introduced. Specifically, the method includes the following steps:
forming a brittle pure-gel conducting hydrogel with a porosity of 20-90%;
infiltrating the pure-gel conducting hydrogel with a secondary stretchable hydrogel to form an interpenetrating double-network hydrogel;
immobilizing ferrocene derivatives on chains of the secondary stretchable hydrogel of the interpenetrating double-network hydrogel through covalent bonds; and
crosslinking glucose oxidases to the secondary stretchable hydrogel of the interpenetrating double-network hydrogel utilizing a room-temperature crosslinker to obtain a stretchable, conducting, and redox-active hydrogel.

In accordance with one embodiment of the present invention, the brittle pure-gel conducting hydrogel is PEDOT:PSS.

In accordance with one embodiment of the present invention, the stretchable hydrogel is selected from PAAm, PAAc or GelMA.

In accordance with one embodiment of the present invention, the room-temperature crosslinker is PEGDE.

In accordance with one embodiment of the present invention, the ferrocene derivatives comprise ferrocenium ion, ferrocene carboxylic acid, ferrocene methanol, ferrocenylmethyl trimethylammonium, ferrocene boronic acid, and ferrocene dimethylamine.

In accordance with a third aspect of the present invention, a non-invasive continuous glucose monitoring device configured to continuously measure glucose concentration in an analyte and output a data stream associated with glucose concentration is presented. The continuous glucose monitoring device includes:
a stretchable, conducting, and redox-active membrane, including:
   an interpenetrating double-network structure, comprising:
      a brittle pure-gel conducting hydrogel; and
      a stretchable hydrogel, wherein the stretchable hydrogel infiltrates the brittle pure-gel conducting hydrogel to form the interpenetrating double-network structure; wherein ferrocene derivatives are immobilized on chains of the stretchable hydrogel via covalent bonds and glucose oxidases are crosslinked to the stretchable hydrogel utilizing a room-temperature crosslinker; and
a current sensor configured for detecting an electric current generated by the membrane via the electrochemical oxidation of hydrogen peroxide (H₂O₂).

In accordance with one embodiment of the present invention, the device further includes at least one processor configured to process the data stream from the non-invasive continuous glucose monitoring device.

In accordance with one embodiment of the present invention, a user interface is configured to display measured glucose concentration values.

In accordance with one embodiment of the present invention, the user interface is integrated into a smartphone application.

In accordance with one embodiment of the present invention, the device further includes an alarm, wherein the alarm is configured to warn a user of a pending hyperglycemic event or a pending hypoglycemic event.

In accordance with one embodiment of the present invention, the device further includes a cloud system configured to save the data stream and user preference settings.

In accordance with one embodiment of the present invention, the device further includes a wireless communication module configured to transmit glucose concentration data to an external device.

### BRIEF DESCRIPTION OF THE DRAWINGS:

Embodiments of the invention are described in more detail hereinafter with reference to the drawings, in which:

FIG. 1 depicts an illustration of a stretchable, conducting, and redox-active hydrogel in accordance with one embodiment of the present invention;

FIG. 2 depicts the steps of fabricating a stretchable, conducting, and redox-active hydrogel in accordance with one embodiment of the present invention;

FIGS. 3A-3C depicts the characteristics of a stretchable, conducting, and redox-active hydrogel in accordance with one embodiment of the present invention, in which FIG. 3A exhibits the redox properties of the hydrogel, FIG. 3B shows the change of conductivity under cyclic strain between 0% and 200%, FIG. 3C shows the glucose sensitivity of the hydrogel, and FIG. 3D demonstrates the hydrogel's durability; and

FIGS. 4A-4C depict applications of a stretchable, conducting, and redox-active hydrogel according to one embodiment of the present invention, in which FIG. 4A shows a schematic of simple patterning techniques for streamlined electrode and device assembly, FIG. 4B and FIG. 4C demonstrate the use of the hydrogel in body-conformable soft glucose biosensors.

### DETAILED DESCRIPTION

In the following description, devices, materials, and/or fabrication methods related to stretchable, conducting, and redox-active hydrogels and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The term “continuous,” as used herein in reference to analyte sensing, is a broad term and refers without limitation to the continuous, continual, or intermittent (e.g., regular) monitoring of analyte concentration, such as, for example, performing a measurement about every 1 to 10 minutes.

The term “electroactive surface,” as used herein, is a broad term and refers without limitation to the surface of an electrode where an electrochemical reaction is to take place. As one example, in a working electrode, H₂O₂ (hydrogen peroxide) produced by an enzyme-catalyzed reaction of an analyte being detected reacts and thereby creates a measurable electric current. For example, in the detection of glucose, glucose oxidase produces H₂O₂ as a byproduct. The H₂O₂ reacts with the surface of the working electrode to produce two protons (2H⁺), two electrons (2e⁻), and one molecule of oxygen (O₂), which produces the electric current being detected. In the case of the counter electrode, a reducible species, for example, O₂ is reduced at the electrode surface in order to balance the current being generated by the working electrode.

The term “analyte,” as used herein, is a broad term and refers without limitation to a substance or chemical constituent in a biological fluid (for example, blood, interstitial fluid, cerebral spinal fluid, lymph fluid, urine, sweat, saliva, etc.) that can be analyzed. Analytes can include naturally occurring substances, artificial substances, metabolites, or reaction products. In some embodiments, the analyte for measurement by the sensing regions, devices, and methods is glucose.

As used herein, the term “pure gel” refers to a type of hydrogel that is composed of a single, continuous gel phase, characterized by a homogenous and stable network structure. Unlike an unstable two-phase water-gel system, a pure-gel maintains uniform consistency throughout its volume without separating into distinct water and gel phases. This uniformity ensures consistent mechanical and physical properties, making pure gels particularly suitable for applications requiring reliable and predictable performance, such as in biomedical devices, tissue engineering, and drug delivery systems.

In accordance with a first aspect of the present invention, a stretchable, conducting, and redox-active hydrogel is provided. This hydrogel is characterized by its unique interpenetrating double-network structure, which integrates a brittle pure-gel conducting hydrogel with a stretchable hydrogel. The stretchable hydrogel infiltrates the brittle pure-gel conducting hydrogel, forming a cohesive interpenetrating double-network that combines the beneficial properties of both components. The construction of this 3D interpenetrated microstructure is essential to enable efficient electron relay between the enzyme sites and the conducting polymer, directly or via the redox mediator.

In this hydrogel, ferrocene derivatives are immobilized on the chains of the stretchable hydrogel through covalent bonds. This modification is achieved using a room-temperature crosslinker, facilitating the integration of glucose oxidases into the hydrogel matrix. The crosslinking of glucose oxidases ensures the hydrogel's functionality in redox reactions, crucial for applications such as glucose monitoring.

The brittle pure-gel conducting hydrogel network is a critical component of the interpenetrating structure. It can be selected from materials such as poly(3,4-ethylenedioxythiophene) polystyrene sulfonate (PEDOT:PSS), which provides the necessary electrical conductivity while maintaining the integrity of the network. This network is complemented by the stretchable hydrogel, which can be chosen from materials like polyacrylamide (PAAm), poly(acrylic acid) (PAAc), or gelatin methacryloyl (GelMA). These materials are known for their excellent stretchability and mechanical properties, allowing the hydrogel to withstand significant deformation without losing its functionality.

The crosslinker used in the hydrogel formation is a room-temperature crosslinker, such as polyethylene glycol diglycidyl ether (PEGDE). This choice of crosslinker ensures that the crosslinking process occurs under mild conditions, preserving the activity of the incorporated enzymes and the structural integrity of the hydrogel.

The ferrocene derivatives incorporated into the hydrogel include a variety of compounds such as ferrocenium ion, ferrocene carboxylic acid, ferrocene methanol, ferrocenylmethyl trimethylammonium, ferrocene boronic acid, and ferrocene dimethylamine. These derivatives are chosen for their redox activity and compatibility with the hydrogel matrix, enhancing the hydrogel's overall performance in electrochemical applications.

Additionally, the brittle pure-gel conducting hydrogel network possesses a porosity of around 20-90%, tailored to optimize the infiltration of the stretchable hydrogel and the overall performance of the composite material. This porosity is carefully controlled during the hydrogel synthesis process to achieve the desired mechanical and electrical properties.

In summary, the construction and components of a stretchable, conducting, and redox-active hydrogel with an interpenetrating double-network structure are outlined. The integration of ferrocene derivatives and glucose oxidases within this hydrogel matrix results in a multifunctional material with significant potential for use in advanced biosensing and wearable medical devices.

In accordance with a second aspect of the present invention, a method for fabricating a stretchable, conducting, and redox-active hydrogel is introduced. This hydrogel is particularly useful in applications that require flexibility, conductivity, and redox activity, such as in biosensors and wearable medical devices.

The fabrication process begins with the formation of a pure-gel conducting hydrogel. This hydrogel is synthesized with a porosity, ranging from 20 to 90%, that is meticulously controlled to optimize its performance and to facilitate the subsequent infiltration by the secondary hydrogel. The porosity of the pure-gel conducting hydrogel is crucial as it determines the case and extent to which the secondary stretchable hydrogel can penetrate and integrate with the conducting hydrogel, thereby forming a robust interpenetrating double-network structure.

Following the formation of the pure-gel conducting hydrogel, the next step involves infiltrating this hydrogel with a secondary stretchable hydrogel. The stretchable hydrogel infiltrates the conducting hydrogel network, resulting in an interpenetrating double-network hydrogel. This network combines the desirable properties of both hydrogels: the electrical conductivity of the brittle pure-gel conducting hydrogel and the mechanical flexibility of the stretchable hydrogel.

Subsequently, ferrocene derivatives are immobilized on the chains of the secondary stretchable hydrogel within the interpenetrating double-network structure. This immobilization is achieved through covalent bonds, ensuring that the ferrocene derivatives are securely attached to the hydrogel matrix. The choice of ferrocene derivatives, which can include compounds such as ferrocenium ion, ferrocene carboxylic acid, ferrocene methanol, ferrocenylmethyl trimethylammonium, ferrocene boronic acid, and ferrocene dimethylamine, provides the necessary redox activity for the hydrogel.

The final step in the fabrication process involves crosslinking glucose oxidases to the stretchable hydrogels. This crosslinking is facilitated by a room-temperature crosslinker, such as polyethylene glycol diglycidyl ether (PEGDE). The use of a room-temperature crosslinker is advantageous as it preserves the functional integrity of the glucose oxidases and maintains the structural stability of the hydrogel.

The pure-gel brittle conducting hydrogel network used in this method can be selected from materials such as PEDOT:PSS, which offers excellent electrical conductivity and structural rigidity. The secondary stretchable hydrogel can be chosen from materials like PAAm, PAAc or GelMA, known for their exceptional stretchability and compatibility with the conducting hydrogel.

Through this method, a stretchable, conducting, and redox-active hydrogel is obtained. This hydrogel integrates the unique properties of its components, resulting in a material that is both flexible and conductive, with redox activity that is essential for various advanced applications. The fabrication method ensures that the hydrogel is stable, durable, and capable of maintaining its functionality under mechanical stress, making it suitable for use in next-generation biosensing technologies and other medical devices.

In accordance with a third aspect of the present invention, a non-invasive continuous glucose monitoring device designed to continuously measure glucose concentration in an analyte and output a data stream associated with glucose concentration is presented. The device is particularly beneficial for individuals requiring regular monitoring of their glucose levels, such as those with diabetes, as it offers a non-invasive, reliable, and comfortable alternative to traditional glucose monitoring methods.

A key feature of the device is its stretchable, conducting, and redox-active membrane, which enhances its functionality and user comfort. This membrane includes an interpenetrating double-network structure, which includes a brittle pure-gel conducting hydrogel and a stretchable hydrogel. The brittle pure-gel conducting hydrogel network, which can be made from materials such as PEDOT:PSS, provides the necessary electrical conductivity. The stretchable hydrogel, selected from materials like PAAm, PAAc or GelMA, infiltrates the brittle pure-gel conducting hydrogel to form a cohesive interpenetrating double-network structure. This dual- network structure ensures the membrane is both flexible and durable, maintaining its integrity and functionality under mechanical stress.

The membrane further incorporates ferrocene derivatives immobilized on the chains of the stretchable hydrogel via covalent bonds. These ferrocene derivatives, which may include compounds such as ferrocenium ion, ferrocene carboxylic acid, ferrocene methanol, ferrocenylmethyl trimethylammonium, ferrocene boronic acid, and ferrocene dimethylamine, provide the redox-active properties essential for the electrochemical detection of glucose. Glucose oxidases are then crosslinked to the ferrocene derivatives using a room-temperature crosslinker, such as PEGDE. This crosslinking process ensures that the glucose oxidases remain functional and are securely integrated into the membrane.

The device also integrates a current sensor which is responsible for detecting an electric current generated by the electrochemical oxidation of hydrogen peroxide (H₂O₂) happened on the membrane. It is worth noting that the membrane can function independently as a working electrode, eliminating the need for additional electrodes, due to the membrane's inherent electron conductivity. The magnitude of the current detected correlates with the glucose concentration in the body, thereby providing a continuous measurement of glucose levels.

The device also includes a processor configured to process the data stream generated from the glucose concentration measurements. This processed data can be displayed through a user interface, which may be integrated into a smartphone application, offering users easy access to their glucose levels. An alarm feature is included to warn users of pending hyperglycemic or hypoglycemic events, ensuring timely intervention.

Furthermore, the device is equipped with a cloud system for saving the data stream and user preference settings, providing a backup and facilitating long- term monitoring and analysis. A wireless communication module is also integrated into the device, allowing for the transmission of glucose concentration data to external devices, such as smartphones or healthcare providers' systems, enhancing the device's connectivity and usability.

This non-invasive continuous glucose monitoring device represents a significant advancement in the field of glucose monitoring, combining flexibility, durability, and advanced connectivity features to provide a comprehensive solution for continuous glucose measurement.

### EXAMPLES

### Example 1. Fabrication of a Stretchable, Conducting, and Redox-Active Hydrogel

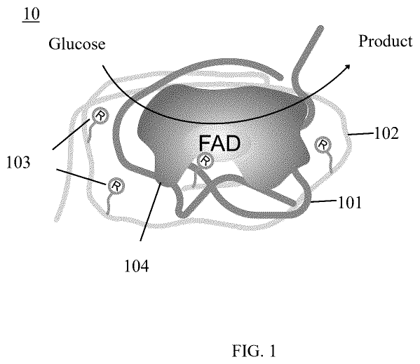

The intrinsically stretchable membrane is developed by combining a conducting polymer PEDOT: PSS hydrogel and a stretchable redox hydrogel which is enzyme-loaded via a covalent bonding approach. As shown in FIG. 1, the hydrogel 10 integrates a brittle pure-gel conducting hydrogel 101 infiltrated with a stretchable hydrogel 102 to form a unique interpenetrating double-network structure. In the hydrogel 10, ferrocene derivatives 103 are immobilized on the chains of the stretchable hydrogel 102 through covalent bonds. Further, a glucose oxidase 104 is integrated into the hydrogel 10 using a room-temperature crosslinker to crosslink the glucose oxidase 104, ensuring the hydrogel's functionality in redox reactions.

The 3D interpenetrated structure strategically utilizes a PEDOT: PSS conducting network to minimize the distance between the enzyme active site (i.e., flavin adenine dinucleotide (FAD)) and the conducting polymers (i.e., electrodes). This design enhances both direct electron transfer (DET) and mediated electron transfer (MET), representing a significant advancement in enzyme technologies for stretchable glucose biosensing.

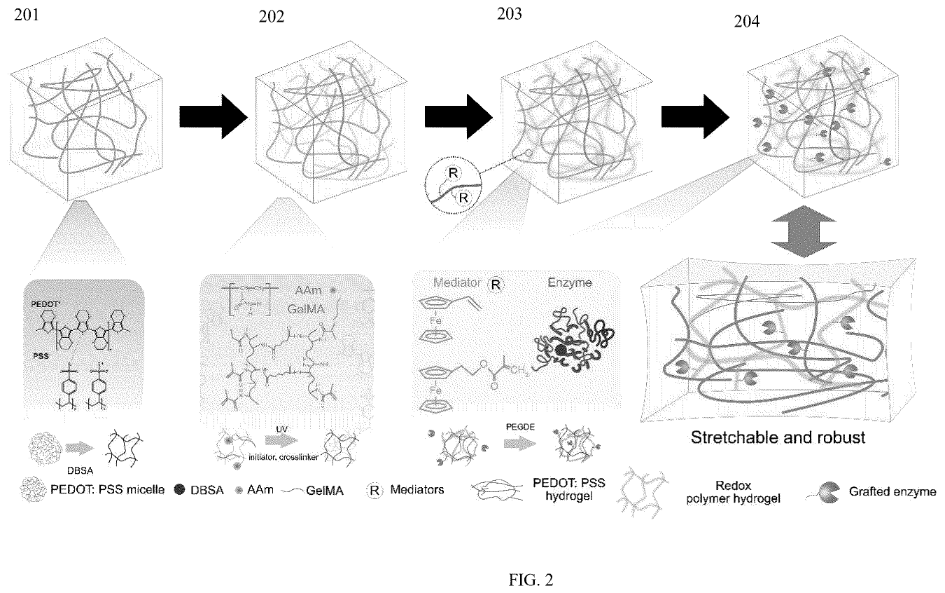

To manufacture the aforementioned stretchable, conducting, and redox-active hydrogel, the steps 201-204 illustrated in FIG. 2 are adopted.

As shown in step 201, the preparation of the hydrogel begins with the synthesis of the PEDOT: PSS brittle pure-gel conducting hydrogel. Briefly, aqueous dispersions of PEDOT: PSS (PH 1000 with weight percentage around 1.1 wt %) are freeze-dried subsequently redispersed to form a high concentration (>3 wt %) suspension by adding various additives to increase the ionic strength of the solution. Notably, increasing the ionic strength of the solution with small molecules such as 4-Dodecylbenzenesulfonic acid (DBSA) can induce gel formation. Increasing the solid concentration from 1.1 wt % to 3 wt % facilitates pure gel formation, avoiding the formation of an unstable two-phase water-gel system. The freeze-drying process significantly enhances the porosity of the conducting network, which is crucial for its interpenetration with the precursors of the secondary stretchable hydrogel network. The successful formation of the PEDOT: PSS hydrogel is confirmed when the mixture's storage modulus (G′) exceeds its loss modulus (G″).

Moving to step 202, to achieve mechanical stretchability, a double-network structure is constructed by infiltrating the brittle pure-gel conducting hydrogel with a secondary stretchable hydrogel network, such as PAAm, PAAc or GelMA. Through the infiltrating and agitation, the secondary hydrogel network forms an interpenetrated secondary network with PEDOT: PSS hydrogel, thus enhancing the overall mechanical properties.

Referring to step 203, ferrocene derivatives are immobilized on the chains of the stretchable hydrogel network to introduce redox and enzymatic functions into the hydrogel. For instance, for PAAm, ferrocene derivatives with vinyl groups (C═C) can form covalent bonds with AAM monomers during the polymerization process.

In the subsequent step 204, glucose oxidase is covalently bonded to the same chains using a room-temperature crosslinker, such as PEGDE. Utilizing room-temperature crosslinking is essential to preserve enzyme activity. Finally, the synthesized stretchable enzyme membrane is purified by washing it in a large amount of distilled water for at least 3 days until the sample reaches equilibrium.

It is noteworthy that tightened hydrogel networks can facilitate electron relay between mediators, enzymes, and conducting polymer chains. For example, interpenetrating with PAAc or calcium-alginate hydrogels can further strengthen the PEDOT: PSS hydrogel. PAAc also contains carboxylic acid groups (COOH) capable of linking with amino groups (NH₂) of the enzymes via COOH—NH₂ bonding.

Additionally, the stretchable enzyme hydrogel can be assembled in different processing sequences. For instance, the enzyme-grafted stretchable hydrogel can serve as the first network, and the monomer 3,4-Ethylenedioxythiophene (EDOT) can be polymerized onto the first network. This method achieves better mechanical stretchability while maintaining enzymatic function.

### Example 2. The Performances of Stretchable, Conducting, and Redox-Active Hydrogel

The intrinsically stretchable hydrogel exhibits electrical conductivity, ionic conductivity, high porosity, redox properties, and glucose-sensing ability within a single material. The hydrogel maintains its conductivity and redox properties even when stretched up to 200% of its initial length. Enhanced catalytic properties are observed after covalently bonding glucose oxidase enzymes to the stretchable redox hydrogel. Due to its inherent electron conductivity, the enzyme membrane can function independently as a working electrode in an electrochemical system, eliminating the need for additional electrodes.

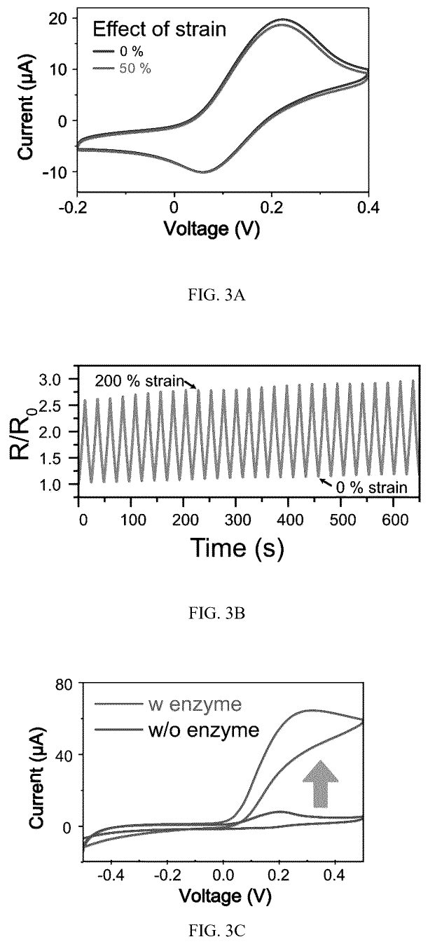

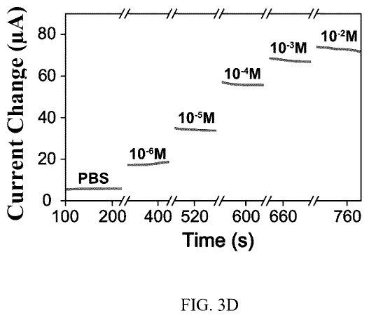

As shown in FIG. 3A, the hydrogel acquires redox properties and becomes strain-robust following the immobilization of ferrocene derivatives. The electromechanical performance of such hydrogel is also investigated. As shown in FIG. 3B, the change of conductivity under cyclic strain between 0% and 200% is depicted, suggesting that the hydrogel is still highly durable after intensive stretched.

As illustrated in FIG. 3C, the hydrogel becomes sensitive to glucose upon the immobilization of glucose oxidase. Referring to FIG. 3D, the membrane can maintain linear sensitivity to glucose concentrations ranging from 10⁻⁶ M to 10⁻² M even after being rinsed in water for one week, which is attributable to the covalent bonds formed between the enzymes and the hydrogel.

### Example 3. The Applications of the Stretchable, Conducting, and Redox-Active Hydrogel

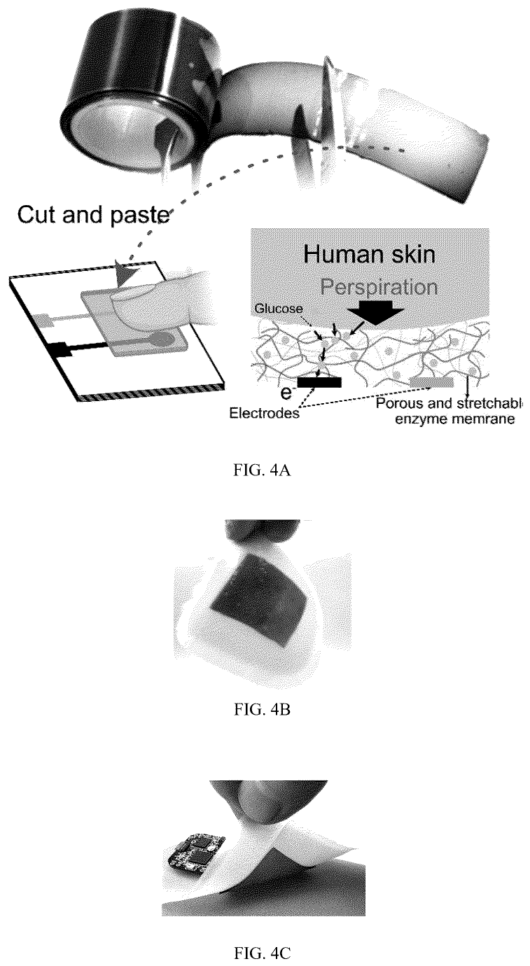

The fabricated hydrogel can be processed using a simple “cut-and-stick” patterning method for device fabrication, which is easier for manual operation and more favorable for large-scale production than conventional solution-based patterning techniques. A schematic of the tape-like “cut-and-stick” patterning is depicted in FIG. 4A. Initially, a free-standing hydrogel membrane is formed. Next, the hydrogel membrane is delaminated and handled like tape. It can be cut into arbitrary shapes using a razor blade, scissors, or stamp, and then attached to an arbitrarily shaped substrate, whether flat or curved. Due to its mechanical stretchability and robustness, cracking does not occur during these cut and transfer processes.

Furthermore, the hydrogel membrane can easily gain adhesive properties by leveraging the abundant dynamic bonds available in the hydrogel composite or by introducing additional dynamic bonds. For example, during the polymerization of the secondary network, the backbone can be modified with N-hydroxysuccinimide (NHS), which reacts effectively with the primary amino group (—NH₂) of the tissue via NHS-NH₂ bonding, thus contributing to high adhesion with human skin. By doing so, the hydrogel membrane is made adhesive, ensuring more reliable sensing on the skin. In parallel, these stretchable enzymatic hydrogel membranes can be easily laminated onto a glass or plastic substrate to assemble novel touch-based glucose sensors. Compared to current methods for enzyme membrane fabrication in the industry, the proposed cut-and-paste approach will markedly simplify the manufacturing process, thus improving the commercial potential for large-scale production while reducing device-to-device variations.

Furthermore, the hydrogel is highly practical for applications in wearable biosensing devices. Stretchable and resilient body-integrated biosensors have recently garnered significant attention for non-invasive continuous glucose monitoring. With its intrinsic stretchability and the case of patterning, the hydrogel membranes of the present invention are well-suited for developing emerging intrinsically stretchable and tissue-conformable medical wearables. These include touch-based glucose sensors, sweat-sensing patches, and wound-monitoring bandages, as illustrated in FIG. 4B and FIG. 4C.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A stretchable, conducting, and redox-active hydrogel, comprising:
an interpenetrating double-network structure, comprising:
a brittle pure-gel conducting hydrogel; and
a stretchable hydrogel, wherein the stretchable hydrogel infiltrates the brittle pure-gel conducting hydrogel to form the interpenetrating double-network structure;
wherein ferrocene derivatives are immobilized on chains of the stretchable hydrogel via covalent bonds and glucose oxidases are crosslinked to the stretchable hydrogel utilizing a room-temperature crosslinker.

2. The hydrogel of claim 1, wherein the hydrogel remains conductive and maintains redox properties after stretching up to 200% of the initial length.

3. The hydrogel of claim 1, wherein the brittle pure-gel conducting hydrogel network is poly(3,4-ethylenedioxythiophene) polystyrene sulfonate (PEDOT:PSS).

4. The hydrogel of claim 1, wherein the stretchable hydrogel is selected from polyacrylamide (PAAm), poly(acrylic acid) (PAAc), or gelatin methacryloyl (GelMA).

5. The hydrogel of claim 1, wherein the crosslinker is the room-temperature crosslinker is polyethylene glycol diglycidyl ether (PEGDE).

6. The hydrogel of claim 1, wherein the ferrocene derivatives comprise ferrocenium ion, ferrocene carboxylic acid, ferrocene methanol, ferrocenylmethyl trimethylammonium, ferrocene boronic acid, and ferrocene dimethylamine.

7. The hydrogel of claim 1, wherein the brittle pure-gel conducting hydrogel has a porosity ranging from 20% to 90%.

8. A method of fabricating a stretchable, conducting, and redox-active hydrogel, comprising:
forming a pure-gel conducting hydrogel with a porosity of 20-90%;
infiltrating the pure-gel conducting hydrogel with a secondary stretchable hydrogel to form an interpenetrating double-network hydrogel;
immobilizing ferrocene derivatives on chains of the secondary stretchable hydrogel of the interpenetrating double-network hydrogel through covalent bonds; and
crosslinking glucose oxidases to the ferrocene derivatives utilizing a room-temperature crosslinker to obtain a stretchable, conducting, and redox-active hydrogel.

9. The method of claim 8, wherein the brittle pure-gel conducting hydrogel network is PEDOT:PSS.

10. The method of claim 8, wherein the stretchable hydrogel is selected from PAAm or PAAc.

11. The method of claim 8, the crosslinker is the room-temperature crosslinker is PEGDE.

12. The method of claim 8, wherein the ferrocene derivatives comprise ferrocenium ion, ferrocene carboxylic acid, ferrocene methanol, ferrocenylmethyl trimethylammonium, ferrocene boronic acid, and ferrocene dimethylamine.

13. A non-invasive continuous glucose monitoring device configured to continuously measure glucose concentration in an analyte and output a data stream associated with glucose concentration, wherein the continuous glucose monitoring device comprises:
a stretchable, conducting, and redox-active membrane, comprising:
an interpenetrating double-network structure, comprising:
a brittle pure-gel conducting hydrogel; and
a stretchable hydrogel, wherein the stretchable hydrogel infiltrates the brittle pure-gel conducting hydrogel to form the interpenetrating double-network structure;
wherein ferrocene derivatives are immobilized on chains of the stretchable hydrogel via covalent bonds and glucose oxidases are crosslinked to the ferrocene derivatives utilizing a room-temperature crosslinker; and
a current sensor configured for detecting an electric current generated by the membrane via the electrochemical oxidation of hydrogen peroxide (H₂O₂).

14. The non-invasive continuous glucose monitoring device of claim 13, further comprising at least one processor configured to process the data stream from the non-invasive continuous glucose monitoring device.

15. The non-invasive continuous glucose monitoring device of claim 13, further comprising a user interface configured to display measured glucose concentration values.

16. The non-invasive continuous glucose monitoring device of claim 15, wherein the user interface is integrated into a smartphone application.

17. The non-invasive continuous glucose monitoring device of claim 13, further comprising an alarm, wherein the alarm is configured to warn a user of a pending hyperglycemic event or a pending hypoglycemic event.

18. The non-invasive continuous glucose monitoring device of claim 13, further comprising a cloud system configured to save the data stream and user preference settings.

19. The non-invasive continuous glucose monitoring device of claim 13, further comprising a wireless communication module configured to transmit glucose concentration data to an external device.
